(19) United States
(12) Patent Application Publication
(10) Pub. No.: US 2012/0165618 A1
(43) Pub. Date: Jun. 28, 2012

(54) METHOD AND APPARATUS FOR HEALTH AVATAR

(76) Inventors: Richard Algoo, Green Brook, NJ (US); Mary George Algoo, Green Brook, NJ (US)

(21) Appl. No.: 13/307,041

(22) Filed: Nov. 30, 2011

Related U.S. Application Data
(60) Provisional application No. 61/425,982, filed on Dec. 22, 2010.

(51) Int. Cl. A61B 5/00

(52) U.S. Cl. 600/300

(57) ABSTRACT

An intelligent health avatar which will aid in the care of patients. The health avatar, through the use of one or more computers executing one or more computer program, and in combination with one or more computer programs, gathers information from patients' health records, patients' monitoring devices, and interactions with patients through computer interfaces. Based on the information gathered, data provided from patients' doctors, and a health rules database, a health avatar, through the use of one or more computer programs, and in combination with one or more computer programs will determine the appropriate course of care for the patient. The health avatar, in at least one embodiment, will interact with the patient in the form of a conversation for formulating patient care options.

### CROSS REFERENCE TO RELATED APPLICATION(S)

This application claims the priority of U.S. provisional patent application Ser. No. 61/425,982, filed on Dec. 22, 2010.

### FIELD OF THE INVENTION

This invention relates to improved methods and apparatus concerning health services.

### BACKGROUND OF THE INVENTION

An avatar is an interactive computer generated image that is a graphical representation of a person. Currently avatars are used primarily in the video game industry to engage users.

### SUMMARY OF THE INVENTION

One or more embodiments of the present invention provide an intelligent health avatar which will aid in the care of patients. The health avatar, through the use of one or more computers executing one or more computer program, and in combination with one or more computer programs, gathers information from patients' health records, patients' monitoring devices, and interactions with patients through computer interfaces. Based on the information gathered, data provided from patients' doctors, and a health rules database, a health avatar, through the use of one or more computer programs, and in combination with one or more computer programs will determine the appropriate course of care for the patient. The health avatar, in at least one embodiment, will interact with the patient in the form of a conversation for formulating patient care options.

### BRIEF DESCRIPTION OF THE DRAWINGS

FIG. 1 shows a block diagram of an apparatus, system, and method for use in accordance with an embodiment of the present invention;

FIG. 2 is a first image which may be produced on a computer monitor of the apparatus of FIG. 1, in accordance with an embodiment of the present invention;

FIG. 3 is a second image which may be produced on a computer monitor of the apparatus of FIG. 1, in accordance with an embodiment of the present invention;

FIG. 4 is a third image which may be produced on a computer monitor of the apparatus of FIG. 1, in accordance with an embodiment of the present invention;

FIG. 5 is a fourth image which may be produced on a computer monitor of the apparatus of FIG. 1, in accordance with an embodiment of the present invention;

FIG. 6 is a fifth image which may be produced on a computer monitor of the apparatus of FIG. 1, in accordance with an embodiment of the present invention;

FIG. 7 is a sixth image which may be produced on a computer monitor of the apparatus of FIG. 1, in accordance with an embodiment of the present invention;

FIG. 8 is a seventh image which may be produced on a computer monitor of the apparatus of FIG. 1, in accordance with an embodiment of the present invention;

FIG. 9 is an eighth image which may be produced on a computer monitor of the apparatus of FIG. 1, in accordance with an embodiment of the present invention;

FIG. 10 is a ninth image which may be produced on a computer monitor of the apparatus of FIG. 1, in accordance with an embodiment of the present invention;

FIG. 11 is a tenth image which may be produced on a computer monitor of the apparatus of FIG. 1, in accordance with an embodiment of the present invention;

FIG. 12 is a eleventh image which may be produced on a computer monitor of the apparatus of FIG. 1, in accordance with an embodiment of the present invention;

FIG. 13 is a twelfth image which may be produced on a computer monitor of a physician interface of the apparatus of FIG. 1, in accordance with an embodiment of the present invention;

FIG. 14 is a thirteenth image which may be produced on a computer monitor of a physician interface of the apparatus of FIG. 1, in accordance with an embodiment of the present invention;

FIG. 15 is a fourteenth image which may be produced on a computer monitor of a physician interface of the apparatus of FIG. 1, in accordance with an embodiment of the present invention;

FIG. 16 is a fifteenth image which may be produced on a computer monitor of a physician interface of the apparatus of FIG. 1, in accordance with an embodiment of the present invention;

FIG. 17 is an sixteenth image which may be produced on a computer monitor of a physician interface of the apparatus of FIG. 1, in accordance with an embodiment of the present invention; and

FIG. 18 is a seventeenth image which may be produced on a computer monitor of a physician interface of the apparatus of FIG. 1, in accordance with an embodiment of the present invention.

### DETAILED DESCRIPTION OF THE DRAWINGS

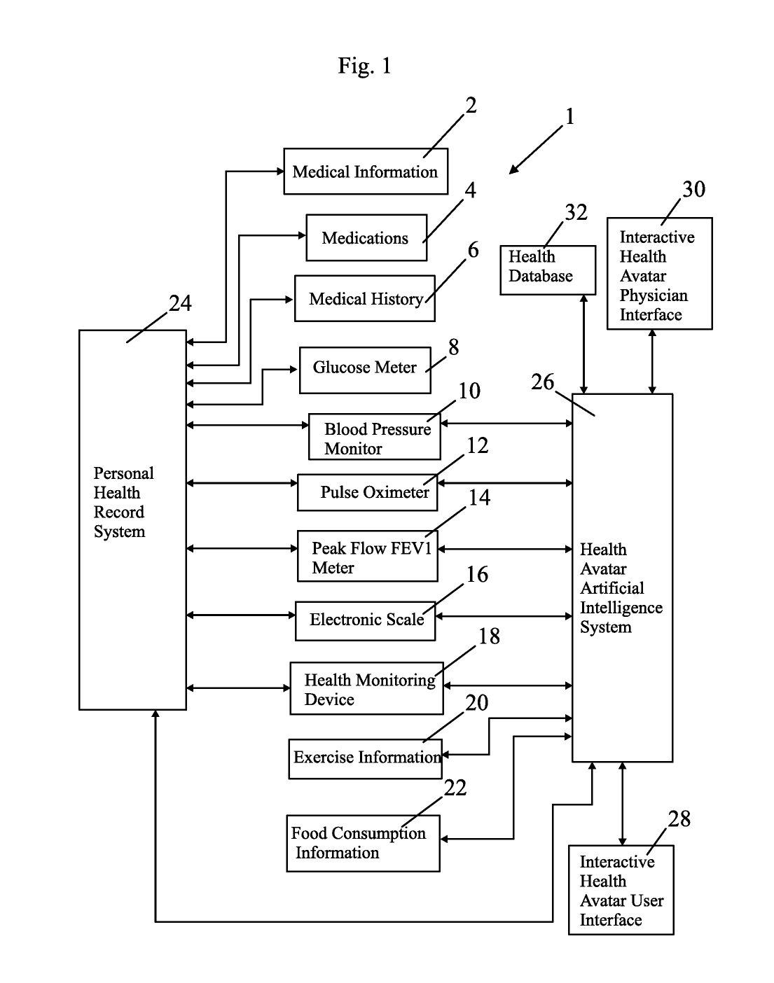

FIG. 1 shows a block diagram 1 of an apparatus, system, and method for use in accordance with an embodiment of the present invention.

The block diagram 1 includes or shows medical information 2, medications 4, medical history 6, glucose meter 8, blood pressure monitor 10, pulse oximeter 12, peak flow FEV1 (“Forced Expiratory Volume in 1 second) meter 14, electronic scale 16, health monitoring device 18, exercise information 20, food consumption information 22, personal health record system 24, health avatar artificial intelligence system 26, and interactive health avatar user interface 28.

Each of the medical information 2, medications 4, medical history 6, glucose meter 8, blood pressure monitor 10, pulse oximeter 12, peak flow FEV1 (”Forced Expiratory Volume in 1 second) meter 14, electronic scale 16, and the health monitoring device 18 are connected by communications links to the personal health record system 24. Each of the blood pressure monitor 10, pulse oximeter 12, peak flow FEV1 (“Forced Expiratory Volume in 1 second) meter 14, electronic scale 16, health monitoring device 18, exercise information 20, food consumption information 22, are connected by a communications link to the health avatar artificial intelligence system 26.

The personal health record system 24 is connected by a communications link to the health avatar artificial intelligence system 26.

Each of the medical information 2, medications 4, medical history 6 may be stored in a single computer or in multiple computers and the components 2, 4, and/or 6 may represent computers and/or databases. The glucose meter 8 may be or may represent any type of known glucose meter which has the ability to transmit data electronically, either through a USB (Universal Serial Bus) connection or wirelessly through WiFi, Bluetooth, or the cellular network to a patient's computer or into a patient's health record. Such devices exist in the health care industry today from manufacturers such as Lifescan (trademarked), and Bayer (trademarked) Diabetes Care. The glucose meter 8 may be controlled by or may provide data to the personal health record system 24, through communications links. The personal health record system 24 may include a computer processor, a computer memory, a computer interface or user interactive device, such as a touch screen, computer mouse or keyboard. The system 24 may also include a computer display or monitor.

The blood pressure monitor 10 may be or may represent any type of known blood pressure monitor which has the ability to transmit data electronically, either through a USB connection or wirelessly through WiFi, Bluetooth, or the cellular network to a patient's computer or into a patient's health record. Such devices exist in the health care industry today from manufacturers such as Omron (trademarked), and A&D Medical (trademarked), and Walgreens (trademarked). The blood pressure monitor 10 may be controlled by or may provide data to the personal health record system 24, through a communications link. The blood pressure monitor 10 may also be controlled by or may provide data to the health avatar artificial intelligence system 26 through a communications link. The health avatar artificial intelligence system 26 may include a computer processor, a computer memory, a computer interface or user interactive device, such as a touch screen, computer mouse or keyboard, and may also include a computer display or monitor.

The pulse oximeter 12 may be or may represent any type of known pulse oximeter, which has the ability to transmit data electronically, either through a USB connection or wirelessly through WiFi, Bluetooth, or the cellular network to a patient's computer or into a patient's health record. Such devices exist in the health care industry today from manufacturers such as Nonin (trademarked). The pulse oximeter 12 may be controlled by or may provide data to the personal health record system 24 through a communications link. The pulse oximeter 12 may also be controlled by or may provide data to the health avatar artificial intelligence system 26 through a communications link.

The peak flow FEV1 meter 14 may be or may represent any type of known peak flow FEV1 meter, which has the ability to transmit data electronically, either through a USB connection or wirelessly through WiFi, Bluetooth, or the cellular network to a patient's computer or into a patient's health record. Such devices exist in the health care industry today from manufacturers such as Microlife (trademarked). The peak flow FEV1 meter 14 may be controlled by or may provide data to the personal health record system 24 through a communications link. The peak flow FEV1 meter 14 may also be controlled by or may provide data to the health avatar artificial intelligence system 26 through a communications link.

The electronic scale 16 may be or may represent any type of known electronic scale device, which has the ability to transmit data electronically, either through a USB connection or wirelessly through WiFi, Bluetooth, or the cellular network to a patient's computer or into a patient's health record. Such devices exist in the health care industry today from manufacturers such as A&D Medical (trademarked), and Withings (trademarked). The electronic scale may be an electronic weight scale for determining an individual or patient's weight. The electronic scale 16 may be controlled by or may provide data to the personal health record system 24 through a communications link. The electronic scale 16 may also be controlled by or may provide data to the health avatar artificial intelligence system 26 through a communications link.

The exercise information 20 may be stored on or may represent a computer having computer memory or computer database having information concerning an individual's exercise habits. The exercise information 20 may be changed by or may provide data to the health avatar artificial intelligence system 26 through a communications link.

The food consumption information 22 may be stored on or may represent a computer having computer memory or a computer database having information concerning an individual's food consumption. The food consumption information 22 may be changed by or may provide data to the health avatar artificial intelligence system 26 through a communications link.

The interactive health avatar user interface 28 may represent and/or may include a computer processor, a computer memory, a computer interface or user interactive device, such as a touch screen, computer mouse or keyboard. The interface 28 may also include a computer display or monitor. Health Rules Database 32 may be a database and/or computer memory of information and data and rules may be stored therein that the health avatar artificial intelligence system 26 may utilize as inputs for making decisions.

In accordance with an embodiment of the present invention, a computer processor of the health avatar artificial intelligence system 26 is programmed by a computer program to access a patient's personal health records, such as through a communications link with the personal health record system 24. The personal health record system 24 may access personal health records through the components 2, 4, and 6.

The health avatar artificial intelligence system 26 is also programmed by a computer program control and receive data from monitoring devices, such as a glucose meter 8, blood pressure meter 10, pulse oximeter 12, peak flow meter FEV1 14, electronic scale 16, and health monitoring device 18 through communications links with those devices. The health avatar artificial intelligence system 26 is also programmed by a computer program to receive data and to update or send data to exercise information 20 and food consumption information 22, each of which may include databases or computer memories.

The health avatar artificial intelligence system 26, in at least one embodiment, utilizes data from the health rules database 32, to formulate patient interactions, patient responses, patient questions, determination of alerts to physicians and other actions taken by the avatar. The health avatar artificial intelligence system 26 may use computer software, neural networking algorithms or other self learning algorithms known in the industry to add to, update, or delete, data and rules in Health Rules Database 32.

The health avatar artificial intelligence system 26 may include audio speakers and/or visual computer monitors and the avatar may provide audio sounds on the speakers or videos on the computer monitors in order to provide information to a patient, as if the system 26 was the patient's doctor, knowing the medical background and current state of health of the patient.

The health avatar artificial intelligence system 26 may be programmed by a computer program to provide personalized information, video instructions, audio instructions, and personalized control of devices 10, 12, 14, 16, and 18 for each patient. The system 26 may be programmed by a computer program ask relevant health questions of a patient by providing audio messages on the audio speakers of the system 26 and/or providing video messages and/or displays on a computer monitor of the health avatar artificial intelligence system 26 or of the interactive health avatar user interface 28. The system 26 may be programmed to provide questions in the form of audio message or video messages which have been personalized for a particular user based on the information system 26 receives from system 24, from devices 10, 12, 14, 16, and 18, and from information sources or computers 20 and 22.

The health avatar artificial intelligence system 26 may be programmed by a computer program to provide recommendations, in the form of audio messages and/or video messages provided by system 26 through for example interface 28, for treatments or activities for the patient to take.

The interface 28 may be or may include personal computer, a mobile device, a smart telephone, a television, or any other device typically having a computer processor, a computer monitor, computer memory, and a computer interactive device such as a computer mouse, keyboard, or touch screen. The health avatar artificial intelligence system 26 may be programmed by a computer program to give each patient expert advice, through audio and/or video messages for that patient's specific, unique, health condition, from the comfort of the patient's home or any other location where the interface 28 may be located.

The health avatar artificial intelligence system or computer 26 may be programmed by a computer program to utilize a medical rules database which may be part system 26 and/or may be stored in computer memory. The system or computer 26 may be programmed by a computer program to be self learning meaning that system 26 may be programmed by a computer program to add new medical rules, delete outdated medical rules, and update existing medical rules, in response to patient feedback on the correctness of Avatar responses to patient questions. The system 26 may be programmed by a computer program to utilize natural language processing, neural networking, and other techniques and technologies to learn and process information, such as provided from personal health record system 24, and from devices or components 10, 12, 14, 16, 18, and sources 20 and 22.

In accordance with at least one embodiment of the present invention, a patient will have access to the health avatar artificial intelligence system 26 anytime, from any location which has access to the internet, typically by use of interactive health avatar user interface 28 connecting through a communications link, which may include the internet, to system 26. The interactive health avatar user interface may be or may include PC (personal computer), mobile device, smart phone or television which has a computer processor and other appropriate hardware and is programmed by a computer program or computer software to access the health avatar artificial intelligence system 26.

In at least one embodiment of the present invention, the health avatar artificial intelligence system 26 is programmed by a computer program to review a patient's health information and monitoring device data, such as received from system 24, devices 10, 12, 14, 16, 18, and sources 20 and 22 and stored in computer memory of the health avatar artificial intelligence system 26. The system 26 may be programmed to review such stored health information and monitoring data, every time the patient logs onto or otherwise engages the health avatar artificial intelligence system 26. If remote monitoring data stored in computer memory of the system 26 is old, the health avatar artificial intelligence system 26 may be programmed by a computer program to either request or obtain new data, such as new data from devices 8, 10, 12, 14, 16, 18, and system 24, and sources 20 and 22. The system 26 may also, through audio message or video messages provided to the interface 28 and displayed on a computer monitor of the interface 28, request that the patient provide current device readings, into an interactive device of interface 28, such as through a computer keyboard, computer mouse, or computer touch screen. These current device readings input by a user using interface 28 may include readings of the type that devices 8, 10, 12, 14, 16, and 18 would provide.

The health avatar artificial intelligence system 26 may be programmed by a computer program to help patients to better manage health at home, which may lead to reduced overall cost of care for patients. Another benefit provided by the system 26 and the system 26 in combination with the other components of apparatus 1 in FIG. 1, is a reduction in questions to the physicians. Patients can as the health avatar artificial intelligence system 26 health related questions, such as by typing a question using a computer keyboard, or inputting with a computer mouse or touch screen of the interface 28, and the health avatar artificial intelligence system 26 may be programmed to receive these questions via a communications link, which may include the internet and to respond with an appropriate answer, by supplying audio messages or video messages to interface 28. The United States of America and worldwide health care systems are greatly stressed and there are not enough doctors to treat the growing patient population. There is anticipated reduction in doctors coming from retirement. The health avatar artificial intelligence system 26 and related components of apparatus 1, may help to remove some of the stress in the health care system.

The health avatar artificial intelligence system 26 and in combination with related components of apparatus 1 will help to reduce hospital re-admissions. The system 26 and in combination with related components of apparatus 1, will give patients more care at home, thereby reducing the number of patients who need to go visit a hospital emergency room. By detecting problems early, the health avatar artificial intelligence system 26 can recommend a doctor visit. This will shift some reactive emergency room visits to proactive managed care.

The health avatar artificial intelligence system 26, and in combination with components of apparatus shown in FIG. 1, will help patients with chronic diseases such as COPD (chronic obstructive pulmonary disease), diabetes, congestive heart failure, and asthma better manage their disease. Implementations of the health avatar artificial intelligence system 26 may be tailored for specific health conditions. Examples include a COPD avatar or computer processor optimized or programmed by a computer program, for treating COPD patients, a diabetes avatar or computer processor optimized or programmed for treating diabetes patients, a CHF avatar programmed or optimized for treating congestive heart failure, and an asthma avatar programmed or optimized for treating asthma.

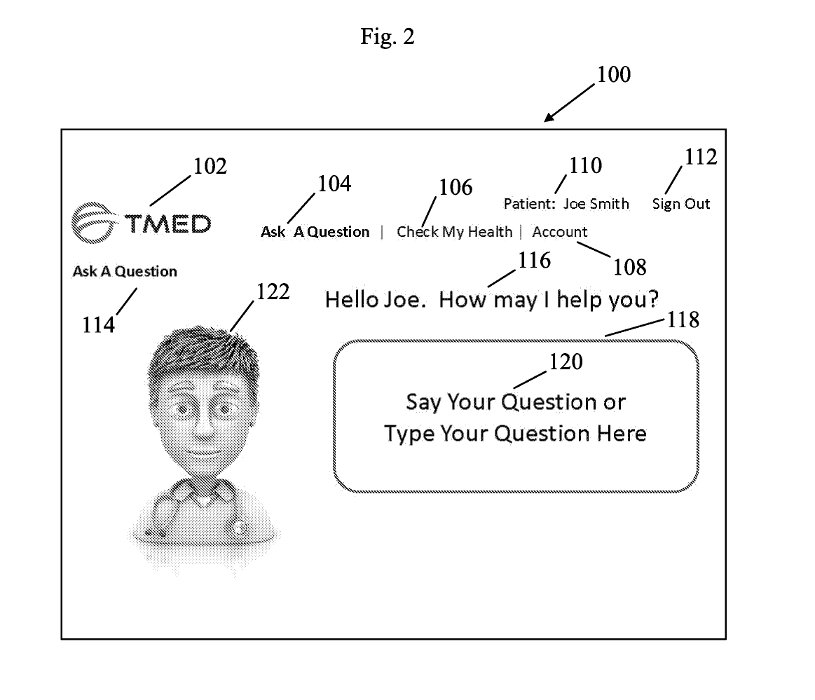

FIG. 2 is a first image 100 which may be produced on a computer monitor of the apparatus 1 of FIG. 1, in accordance with an embodiment of the present invention. For example, the first image 100 may be produced on a computer monitor of the interactive health avatar user interface 28 by a computer program executed by a computer processor of the interface 28 and stored in computer memory of the interface 28. The first image 100 may also be produced by the health avatar artificial intelligence system 26 through a computer program executed by a computer processor of the health avatar artificial intelligence system 26, and stored on computer memory of the system 26. The first image 100 may be provided to the interface 28 by the system 26, through a communications link which may include the internet.

Referring to FIG. 2, the first image 100 includes fields 102, 104, 106, 108, 110, 112, 114, 116, 118, 120, and 122. Field 102 includes text “TMED” and a logo, which may correspond to a company, which may be named “TMED” and its logo, that sponsors, owns, and/or controls the health avatar artificial intelligence system 26 and/or the computer software run on system 26. Field 104 includes text for “Ask A Question”. Field 104 can be selected by a computer interactive device, such as with a computer mouse of interface 28 to go cause the system 26 to cause the image 100 to be provided on the interface 26, such as by providing an internet web page or other data to a computer processor of the interface 26, which is then displayed by a computer processor of the interface 26 on a computer monitor of the interface 26.

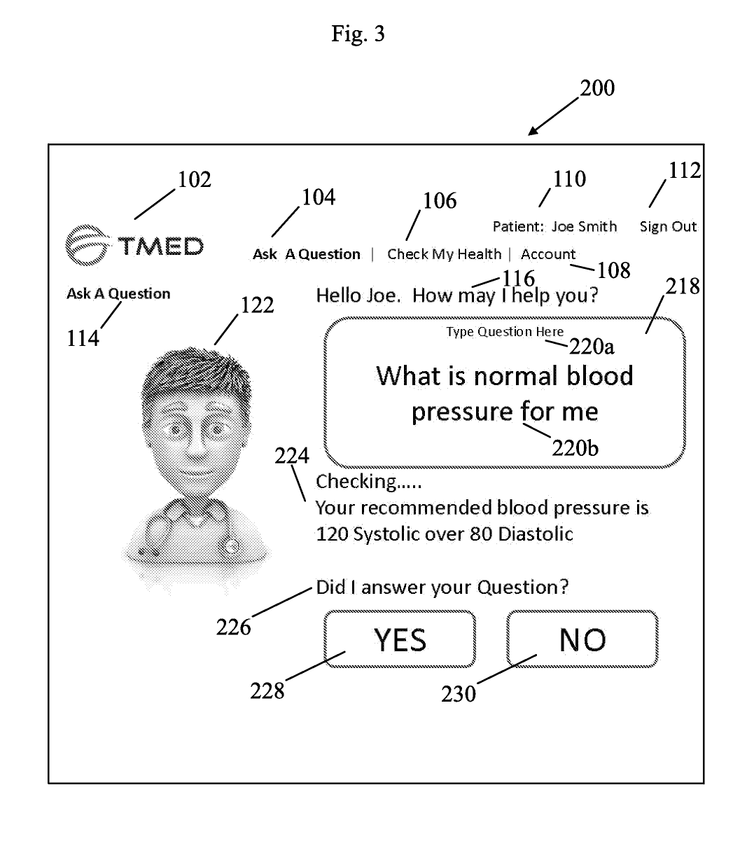

The first image 100 also includes field 106 which includes text “Check My Health” which is a link which can be selected by a computer interactive device, such as with a computer mouse of interface 28 to cause a second image 200 shown in FIG. 3 to be displayed on a computer monitor of the interface 28, such as by being downloaded from the system 26.

The first image 100 also includes field 108 which includes text “Account” which is a link which can be selected by a computer interactive device, such as with a computer mouse of interface 28 to go to a web page, not displayed, where patients can reset password and update personal information such as contact and billing information.

The first image 100 also includes field 110 which includes text “Patient: Joe Smith” which indicates the patient or user that is using the interface 28 and is currently being serviced by system 26.

The first image 100 also includes field 112 which includes text “Sign Out” which is a link which can be selected by a computer interactive device, such as with a computer mouse of interface 28 to sign off of a web site run by the health avatar artificial intelligence system 26.

The first image 100 also includes field 114 which includes text “Ask a Question” which indicates that a web page for “Ask a Question” is currently active, meaning that the “Ask A Question” field 104 was most recently selected by a user, using an interactive device, such as a computer mouse of interface 28 bringing up first image 100 on a computer monitor of interface 28.

The first image 100 also includes field 116 which includes text “Hello Joe. How may I help you?” or some other question which appears to be asked by a field or graphic image or avatar 122. The first image 100 also includes field 118 which includes a box or enclosed area in which is located text 120 “Say Your Question or Type Your Question Here”. The information shown by fields 118 and 120 is located adjacent to the graphic image or avatar 122 so that it appears to come from avatar 122, so the avatar 122 appears to be speaking. A patient or user can user an interactive device, such as a computer keyboard, computer touch screen, computer mouse, or voice recognition device of interface 28 to type in a question, or speak a question out loud to input the question to system 26 through interface 28 and via communications links such as the internet connecting system 26 and interface 28. After the question supplied by interface 28 to system 26 via a communications link, such as including the internet, computer software of the system 26 is programmed in at least one embodiment, to analyze the information and access one or more of system 24, and components 10, 12, 14, 16, 18, 20, and 22 shown in FIG. 1, to determine answers to the particular question. The system 26, includes a computer processor which is programmed to send the answers to the particular question from the system 26 to the interface 28. In at least one embodiment of the present invention, a computer processor of the interface 28 is programmed to display the answers to the particular question on a computer monitor of interface 28.

FIG. 3 is a second image 200 which may be produced on a computer monitor of the apparatus 1 of FIG. 1, in accordance with an embodiment of the present invention. For example, the second image 200 may be produced on a computer monitor of the interactive health avatar user interface 28 by a computer program executed by a computer processor of the interface 28 and stored in computer memory of the interface 28. The second image 200 may also be produced by the health avatar artificial intelligence system 26 through a computer program executed by a computer processor of the health avatar artificial intelligence system 26, and stored on computer memory of the system 26. The second image 200 may be provided to the interface 28 by the system 26, through a communications link which may include the internet.

Referring to FIG. 3, the second image 200 includes fields 102, 104, 106, 108, 110, 112, and 114, 116, 218, 220a, 220b, 122, 224, 226, 228, and 230. Fields 102, 104, 106, 108, 110, 112, 114, 116, and 122 may be similar to or the same as the same numbered fields in FIG. 2.

Field 218 may be an enclosed area or box in which is located the field 220a which includes text “Type Question Here” and the field 220b which includes the text “What is normal blood pressure for me”. The text 220a may be produced in the image 200 on a computer monitor of the interface 28 by one or more computer programs of a computer processor of the system 26 and/or a computer processor of the interface 28. The text 220a may be produced after a person selects the field 104 “Ask a Question”. The text 220b may be typed in or otherwise entered by a user, through a computer interactive device, such as a computer mouse, computer keyboard, voice recognition device, or computer touch screen of the interface 28.

The field 224 may include text “Checking . . . Your recommended blood pressure is 120 Systolic over 80 Diastolic”. The text in field 224 may be produced by a computer program running on the system 26 in response to the question in text 220b. The system 26 may receive data concerning the question 220b from the interface 28 via the internet or other communications links, and may access one or more of system 24 and components 10, 12, 14, 16, 18, 20, and 22 to determine data for an answer to the question 220b. The system 26 may be programmed to provide the answer shown by text in field 224 to the interface 28.

The field 226 may display the question for “Did I answer your Question?” on a computer monitor of the interface 28 by use of computer programs running on the system 26 and the interface 28. The field 228 may include an enclosed area or box and the text for “YES”. The field 230 may include an enclosed area or box and the text for “YES”. The fields 228 and 230, and data shown, may be provided by the system 26 to the interface 28 in response to the information supplied by a user through the interface 28, through the use of a computer program running on the system 26 and stored in computer memory of the system 26. A computer processor of the interface 28 may respond to selection by a user of either fields 228 or 230 by sending information to the system 26 via a communications link to indicate that the user's question has been or has not been answered. Based on the patient's responses to the question “Did I answer your Question?”, the health avatar artificial intelligence system 26, on FIG. 1 may use neural networking algorithms or other self learning algorithms known in the industry to add to, update, or delete, data and rules in Health Rules Database 32.

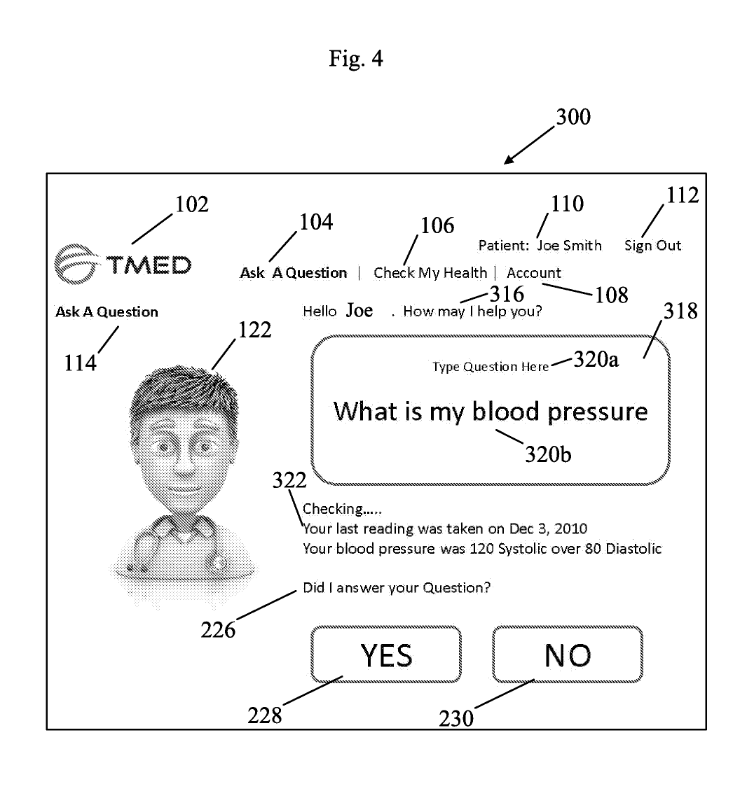

FIG. 4 is a third image 300 which may be produced on a computer monitor of the apparatus 1 of FIG. 1, in accordance with an embodiment of the present invention. For example, the third image 300 may be produced on a computer monitor of the interactive health avatar user interface 28 by a computer program executed by a computer processor of the interface 28 and stored in computer memory of the interface 28. The third image 300 may also be produced by the health avatar artificial intelligence system 26 through a computer program executed by a computer processor of the health avatar artificial intelligence system 26, and stored on computer memory of the system 26. The third image 300 may be provided to the interface 28 by the system 26, through a communications link which may include the internet.

Referring to FIG. 4, the third image 300 includes fields 102, 104, 106, 108, 110, 112, and 114, 316, 318, 320, 122, 322, 226, 228, and 230. Fields 102, 104, 106, 108, 110, 112, 114, and 122 may be similar to or the same as the same numbered fields in FIGS. 2 and 3. Fields 226, 228, and 230 may be similar to or the same as the same numbered fields in FIG. 3.

Field 316 includes text “Hello Joe. How may I help you?” or some other question which appears to be asked by a field or graphic image or avatar 122.

Field 318 may be an enclosed area or box in which is located the text 320a “Type Question Here” and 320b “What is my blood pressure”. The text 320a may be produced in the image 300 on a computer monitor of the interface 28 by one or more computer programs of executed by a computer processor of the system 26 and stored in computer memory of the system 26, and/or executed by a computer processor of the interface 28 and stored in computer memory of the interface 28. The text 320a may be produced after a user or patient selects the field 104 “Ask a Question”. The text 320b may be typed in or otherwise entered by a user, through a computer interactive device, such as a computer mouse, computer keyboard, voice recognition device, or computer touch screen of the interface 28.

The field 322 may include text “Checking . . . Your last reading was taken on Dec. 3, 2010 Your blood pressure was 120 Systolic over 80 Diastolic”. The text in field 322 may be produced by a computer program running on the system 26 in response to the question in text 320b. The system 26 may receive data concerning the question 320b from the interface 28 via the internet or other communications links, and may access one or more of system 24 and components 10, 12, 14, 16, 18, 20, and 22, in response to submission of data regarding the question in text 320b, to determine data for an answer to the question 320b. The system 26 may be programmed by computer software or program, stored in computer memory of the system 26, to provide the answer shown by text in field 324 to the interface 28. Based on the patients responses to the question “Did I answer your Question?”, the health avatar artificial intelligence system 26, on FIG. 1 may use neural networking algorithms or other self learning algorithms know in the industry to add to, update, or delete, data and rules in Health Rules Database 32.

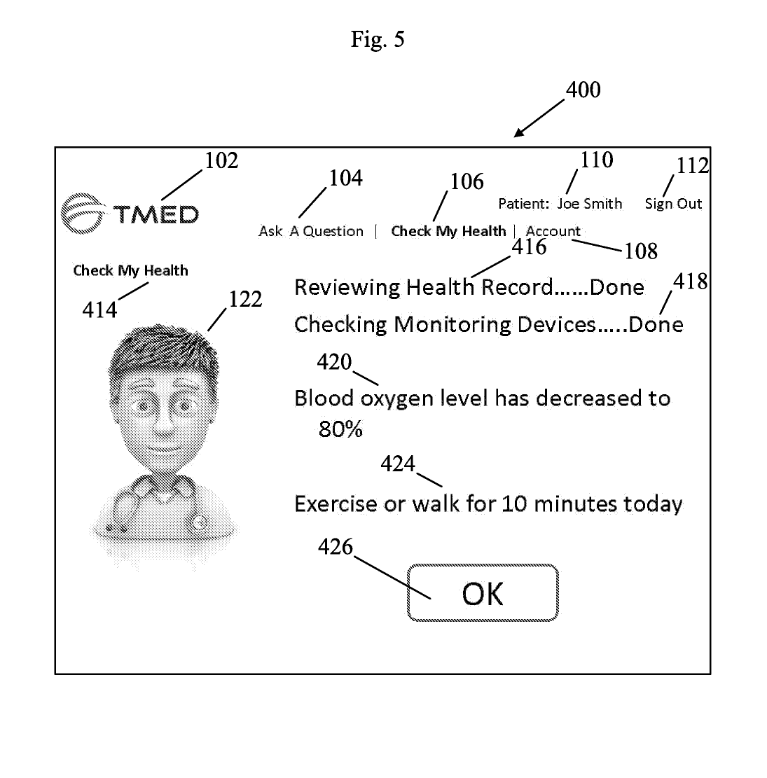

FIG. 5 is a fourth image 400 which may be produced on a computer monitor of the apparatus 1 of FIG. 1, in accordance with an embodiment of the present invention. For example, the fourth image 300 may be produced on a computer monitor of the interactive health avatar user interface 28 by a computer program executed by a computer processor of the interface 28 and stored in computer memory of the interface 28. The fourth image 400 may also be produced by the health avatar artificial intelligence system 26 through a computer program executed by a computer processor of the health avatar artificial intelligence system 26, and stored on computer memory of the system 26. The fourth image 400 may be provided to the interface 28 by the system 26, through a communications link which may include the internet.

Referring to FIG. 5, the fourth image 400 includes fields 102, 104, 106, 108, 110, 112, and 414, 416, 418, 420, 122, 424, and 426. Fields 102, 104, 106, 108, 110, 112, and 122 may be similar to or the same as the same numbered fields in FIGS. 2, 3, and 4.

Field 414 includes text “Check My Health” which indicates that the system 28 has provided a “Check My Health” web page or data to the interface 26 through a communications link such as the internet. The field 414 may be displayed on a computer monitor of the interface 26. Field 416 includes text “Reviewing Health Record . . . . Done”. The text in field 416 may indicate that information from personal health record system 24 has been received by the system 28, and that a computer processor of the system 28 has completed receiving and reviewing the information from the system 24.

Field 418 includes text “Checking Monitoring Devices . . . Done”. The text in field 418 may indicate that the system 28 has finished receiving data from and/or checking devices 10, 12, 14, 16, 18, 20, and 22 of FIG. 1.

Field 420 includes text “Blood oxygen level has decreased to 80%”. Field 424 includes text “Exercise or walk for 10 minutes today”. Field 426 includes an enclosed area or box and the text “OK”. The fields 416 and 418 provide status information on a computer monitor of the interface 28. The information of fields 416 and 418 is typically provided by system 26 through a computer program running on system 26 and stored in computer memory of system 26, via the internet to a computer monitor of interface 28. Field 420 provides information concerning the patient and field 424 provides advice to the patient. The field 426 can be selected by a user using a computer interactive device of the interface 28 to indicate that a user has seen the information on the image 400 of FIG. 5.

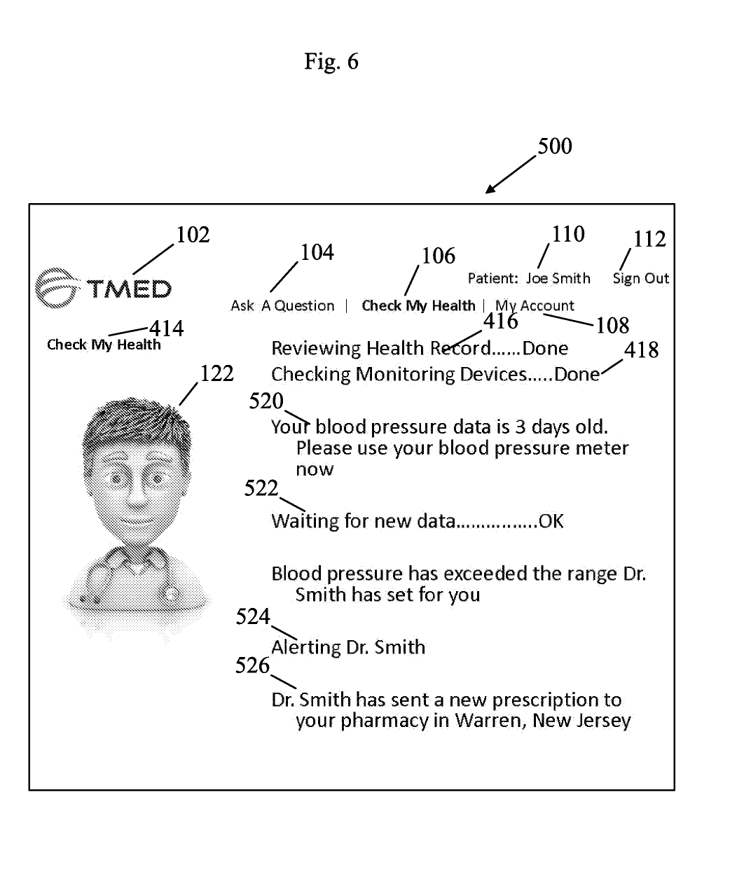

FIG. 6 is a fifth image 500 which may be produced on a computer monitor of the apparatus 1 of FIG. 1, in accordance with an embodiment of the present invention. For example, the fifth image 500 may be produced on a computer monitor of the interactive health avatar user interface 28 by a computer program executed by a computer processor of the interface 28 and stored in computer memory of the interface 28. The fifth image 500 may also be produced by the health avatar artificial intelligence system 26 through a computer program executed by a computer processor of the health avatar artificial intelligence system 26, and stored on computer memory of the system 26. The fifth image 500 may be provided to the interface 28 by the system 26, through a communications link which may include the internet.

Referring to FIG. 6, the fifth image 500 includes fields 102, 104, 106, 108, 110, 112, and 414, 416, 418, 520, 522, 524, and 526. Fields 102, 104, 106, 108, 110, 112, and 122 may be similar to or the same as the same numbered fields in FIGS. 2, 3, and 4. Fields 414, 416, and 418 may be similar to or the same as the same numbered fields in FIG. 4.

Field 520 includes text “Your blood pressure data is 3 days old. Please use your blood pressure meter now”. Field 522 includes text “Waiting for new data OK”. Field 524 includes text “Alerting Dr. Smith”, and field 526 includes text “Dr. Smith has sent a new prescription to your pharmacy in Warren, N.J.”. Fields 520, 522, 524, and 526 may be provided by system 26 to interface 28 after system 26 has received information from system 24 and devices 10, 12, 14, 16, 18, 20, and 22 and in response to selecting field 414 for “Check My Health” and in response to computer algorithms in the Health Avatar Artificial Intelligence system, and the data from the Health Rules Database, which send an alert to the physicians interface, where the physician has approved a new prescriptions.

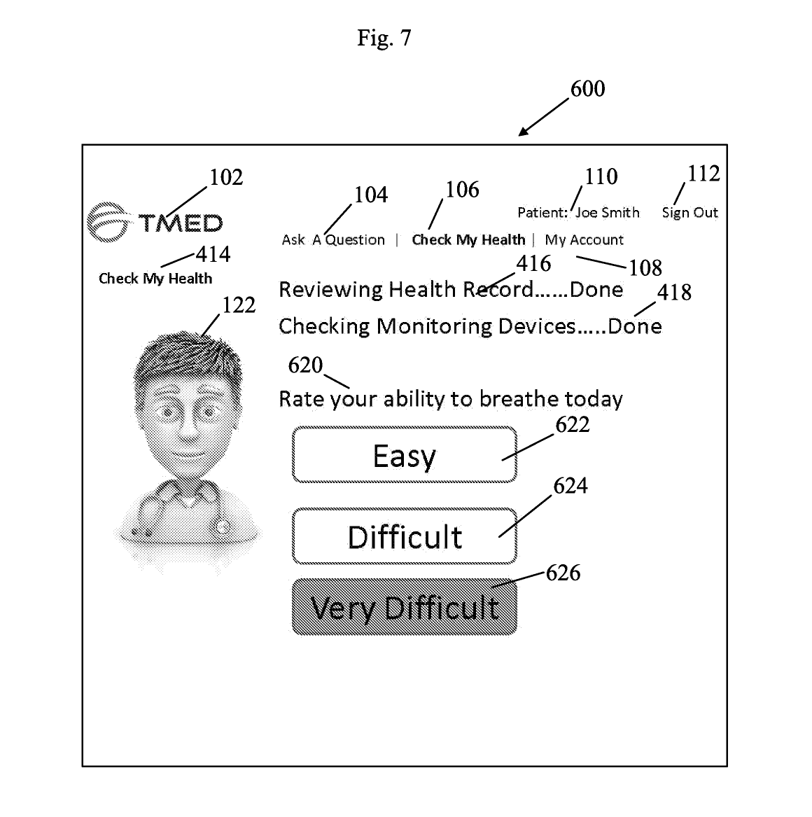

FIG. 7 is a sixth image 600 which may be produced on a computer monitor of the apparatus 1 of FIG. 1, in accordance with an embodiment of the present invention. For example, the sixth image 600 may be produced on a computer monitor of the interactive health avatar user interface 28 by a computer program executed by a computer processor of the interface 28 and stored in computer memory of the interface 28. The sixth image 600 may also be produced by the health avatar artificial intelligence system 26 through a computer program executed by a computer processor of the health avatar artificial intelligence system 26, and stored on computer memory of the system 26. The sixth image 600 may be provided to the interface 28 by the system 26, through a communications link which may include the internet.

Referring to FIG. 7, the sixth image 600 includes fields 102, 104, 106, 108, 110, 112, 122, 414, 416, 418, 620, 622, 624, and 626. Fields 102, 104, 106, 108, 110, 112, and 122 may be similar to or the same as the same numbered fields in FIG. 1. Fields 414, 416, and 418 may be similar to or the same as the same numbered fields in FIG. 4.

Field 620 includes text “Rate your ability to breathe today”. Field 622 includes an enclosure or box with the text “Easy”, field 624 includes an enclosure or box with the text “Difficult”, and field 626 includes an enclosure or box with the text “Very Difficult”. Any one of the fields or buttons 622, 624, and 626 can be selected by a user, using a computer interactive device of the interface 28 to rate the user's breathing. A computer processor of the interface 28 may be programmed to process the selection of one of the buttons 622, 624, and 626 and supply data relating thereto, to the system 26 via a communications link such as including the internet. The computer processor of the interface 28 may execute a web page or program relating to image 600, which is provided from or downloaded from system 26. The health avatar artificial intelligence system 26, in at least one embodiment, utilizes the patients response, and data from the Health Rules Database 32, to formulate the next patient interactions, patient questions, or determination of alerts to physicians and other actions taken by the avatar.

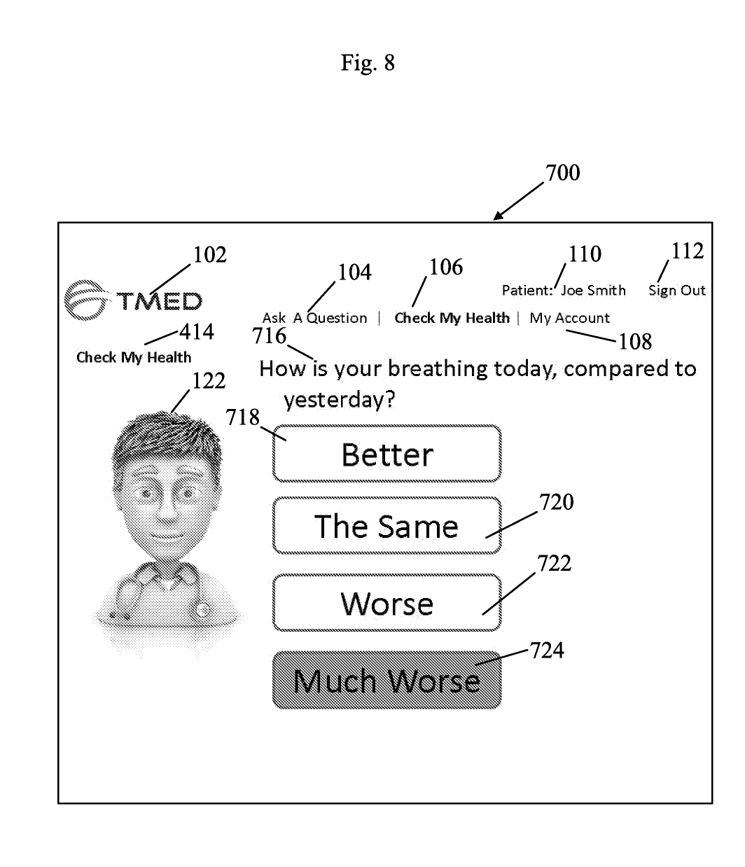

FIG. 8 is a seventh image 700 which may be produced on a computer monitor of the apparatus 1 of FIG. 1, in accordance with an embodiment of the present invention. For example, the seventh image 700 may be produced on a computer monitor of the interactive health avatar user interface 28 by a computer program executed by a computer processor of the interface 28 and stored in computer memory of the interface 28. The seventh image 700 may also be produced by the health avatar artificial intelligence system 26 through a computer program executed by a computer processor of the health avatar artificial intelligence system 26, and stored on computer memory of the system 26. The seventh image 700 may be provided to the interface 28 by the system 26, through a communications link which may include the internet.

Referring to FIG. 8, the seventh image 700 includes fields 102, 104, 106, 108, 110, 112, 414, 122, 716, 718, 720, 722, and 724. Fields 102, 104, 106, 108, 110, 112, and 122 may be similar to or the same as the same numbered fields in FIG. 1. Field 414 may be similar to or the same as the same numbered field in FIG. 4.

Field 716 includes text “How is your breathing today, compared to yesterday”. Field 718 includes an enclosure or box with text “Better, field 720 includes an enclosure or box with text “The Same”, field 722 includes an enclosure or box with text “Worse”, and field 724 includes an enclosure or box with text “Much Worse”. Any one of the fields or buttons 718, 720, 722, and 724 can be selected by a user, using a computer interactive device of the interface 28 to answer the question of field 716. A computer processor of the interface 28 may be programmed to process the selection of one of the buttons 718, 720, 722, and 724 and supply data relating thereto, to the system 26 via a communications link such as including the internet. The computer processor of the interface 28 may execute a web page or program relating to image 700, which is provided from or downloaded from system 26. The health avatar artificial intelligence system 26, in at least one embodiment, utilizes the patients response, and data from the Health Rules Database 32, to formulate the next patient interactions, patient questions, or determination of alerts to physicians and other actions taken by the avatar.

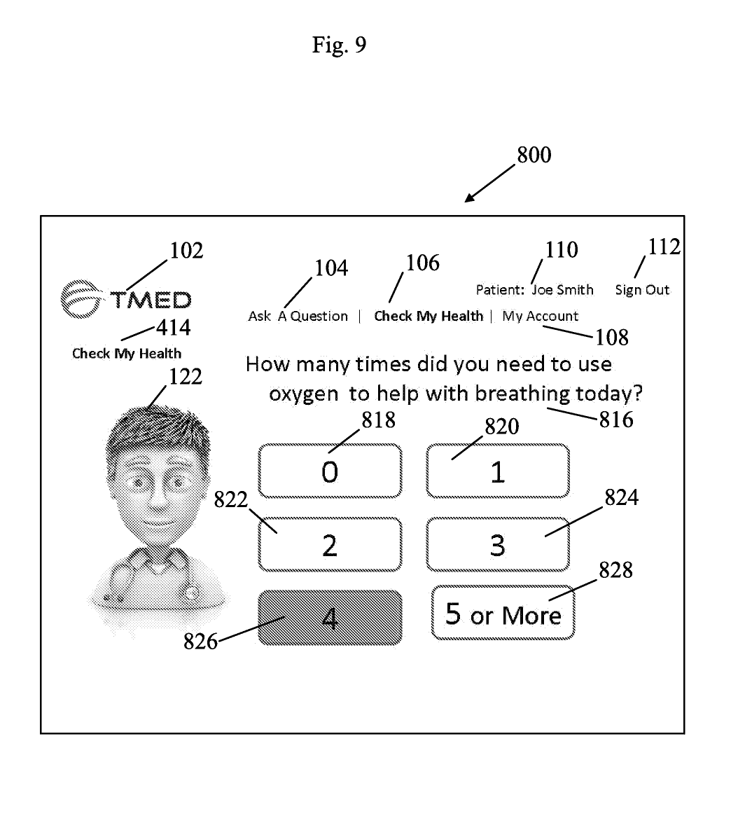

FIG. 9 is an eighth image 800 which may be produced on a computer monitor of the apparatus 1 of FIG. 1, in accordance with an embodiment of the present invention. For example, the eighth image 800 may be produced on a computer monitor of the interactive health avatar user interface 28 by a computer program executed by a computer processor of the interface 28 and stored in computer memory of the interface 28. The eighth image 800 may also be produced by the health avatar artificial intelligence system 26 through a computer program executed by a computer processor of the health avatar artificial intelligence system 26, and stored on computer memory of the system 26. The eighth image 800 may be provided to the interface 28 by the system 26, through a communications link which may include the internet.

Referring to FIG. 9, the eighth image 800 includes fields 102, 104, 106, 108, 110, 112, 414, 122, 816, 818, 820, 822, 824, 826, and 828. Fields 102, 104, 106, 108, 110, 112, and 122 may be similar to or the same as the same numbered fields in FIG. 1. Field 414 may be similar to or the same as the same numbered field in FIG. 4.

Field 816 includes text “How many times did you need to use oxygen to help with breathing today?”. Each of fields 818, 820, 822, 824, 826, and 828 include enclosed areas or boxes. Fields 818, 820, 822, 824, 826, and 828 include text “0”, “1”, “2”, “3”, “4”, and “5 or more”, respectively, which indicate an answer to the question in field 816. Any one of the fields or buttons 818, 820, 822, and 824 can be selected by a user, using a computer interactive device of the interface 28 to answer the question of field 816. A computer processor of the interface 28 may be programmed to process the selection of one of the buttons 818, 820, 822, and 824 and supply data relating thereto, to the system 26 via a communications link such as including the internet. The computer processor of the interface 28 may execute a web page or program relating to image 800, which is provided from or downloaded from system 26. The health avatar artificial intelligence system 26, in at least one embodiment, utilizes the patients response, and data from the Health Rules Database 32, to formulate the next patient interactions, patient questions, or determination of alerts to physicians and other actions taken by the avatar.

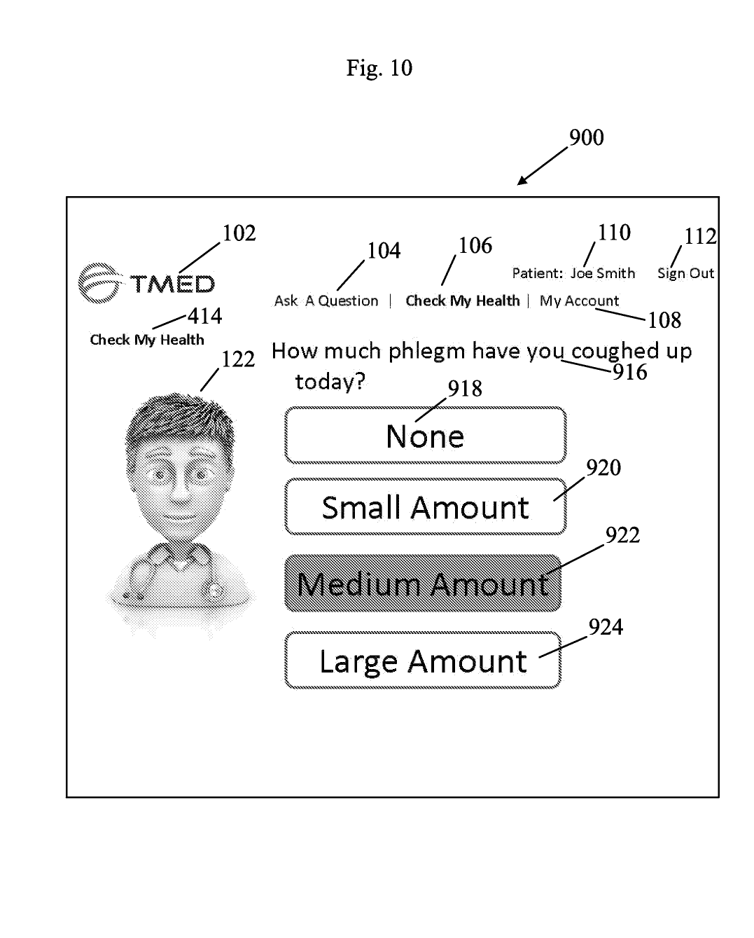

FIG. 10 is an ninth image 900 which may be produced on a computer monitor of the apparatus 1 of FIG. 1, in accordance with an embodiment of the present invention. For example, the eighth image 900 may be produced on a computer monitor of the interactive health avatar user interface 28 by a computer program executed by a computer processor of the interface 28 and stored in computer memory of the interface 28. The ninth image 900 may also be produced by the health avatar artificial intelligence system 26 through a computer program executed by a computer processor of the health avatar artificial intelligence system 26, and stored on computer memory of the system 26. The ninth image 900 may be provided to the interface 28 by the system 26, through a communications link which may include the internet.

Referring to FIG. 10, the ninth image 900 includes fields 102, 104, 106, 108, 110, 112, 414, 122, 916, 918, 920, 922, and 924. Fields 102, 104, 106, 108, 110, 112, and 122 may be similar to or the same as the same numbered fields in FIG. 1. Field 414 may be similar to or the same as the same numbered field in FIG. 4.

Field 916 includes text “How much phlegm have you coughed up today?”. Each of fields 918, 920, 922, and 924 include enclosed areas or boxes. Fields 918, 920, 922, and 924 include text “None”, “Small Amount”, “Medium Amount”, and “Large Amount”, respectively, which indicate an answer to the question in field 916. Any one of the fields or buttons 918, 920, 922, and 924 can be selected by a user, using a computer interactive device of the interface 28 to answer the question of field 916. A computer processor of the interface 28 may be programmed to process the selection of one of the buttons 918, 920, 922, and 924 and supply data relating thereto, to the system 26 via a communications link such as including the internet. The computer processor of the interface 28 may execute a web page or program relating to image 900, which is provided from or downloaded from system 26. The health avatar artificial intelligence system 26, in at least one embodiment, utilizes the patients response, and data from the Health Rules Database 32, to formulate the next patient interactions, patient questions, or determination of alerts to physicians and other actions taken by the avatar.

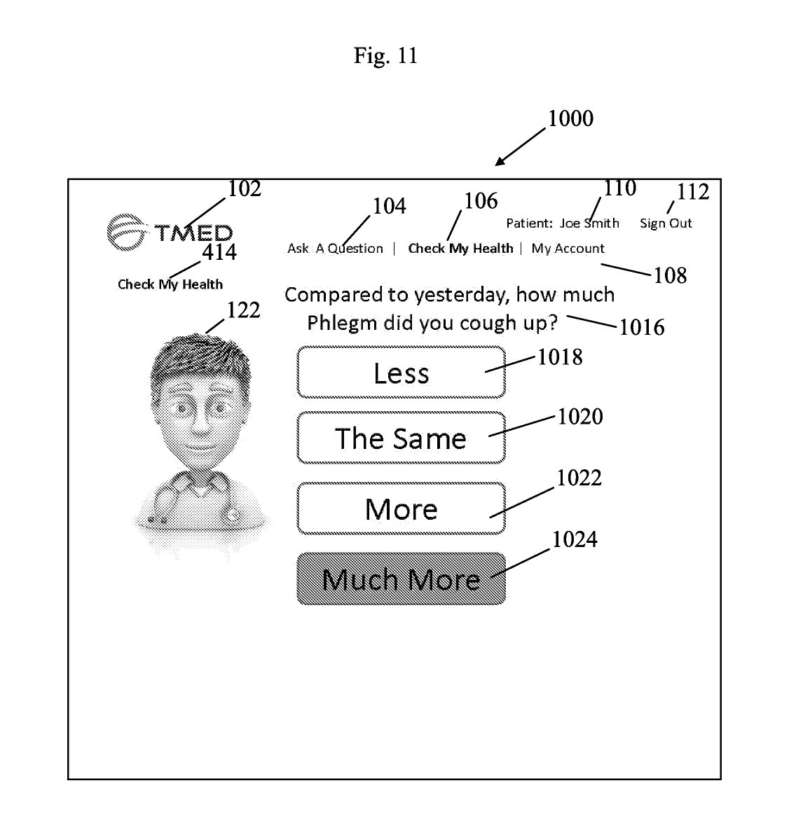

FIG. 11 is an tenth image 1000 which may be produced on a computer monitor of the apparatus 1 of FIG. 1, in accordance with an embodiment of the present invention. For example, the eighth image 1000 may be produced on a computer monitor of the interactive health avatar user interface 28 by a computer program executed by a computer processor of the interface 28 and stored in computer memory of the interface 28. The tenth image 1000 may also be produced by the health avatar artificial intelligence system 26 through a computer program executed by a computer processor of the health avatar artificial intelligence system 26, and stored on computer memory of the system 26. The tenth image 1000 may be provided to the interface 28 by the system 26, through a communications link which may include the internet.

Referring to FIG. 11, the tenth image 1000 includes fields 102, 104, 106, 108, 110, 112, 414, 122, 1016, 1018, 1020, 1022, and 1024. Fields 102, 104, 106, 108, 110, 112, and 122 may be similar to or the same as the same numbered fields in FIG. 1. Field 414 may be similar to or the same as the same numbered field in FIG. 4.

Field 1016 includes text “Compared to yesterday, how much Phlegm did you cough up?”. Each of fields 1018, 1020, 1022, and 1024 include enclosed areas or boxes. Fields 1018, 1020, 1022, and 1024 include text “Less”, “The Same”, “More”, and “Much More”, respectively, which indicate an answer to the question in field 1016. Any one of the fields or buttons 1018, 1020, 1022, and 1024 can be selected by a user, using a computer interactive device of the interface 28 to answer the question of field 1016. A computer processor of the interface 28 may be programmed to process the selection of one of the buttons 1018, 1020, 1022, and 1024 and supply data relating thereto, to the system 26 via a communications link such as including the internet. The computer processor of the interface 28 may execute a web page or program relating to image 1000, which is provided from or downloaded from system 26. The health avatar artificial intelligence system 26, in at least one embodiment, utilizes the patients response, and data from the Health Rules Database 32, to formulate the next patient interactions, patient questions, or determination of alerts to physicians and other actions taken by the avatar.

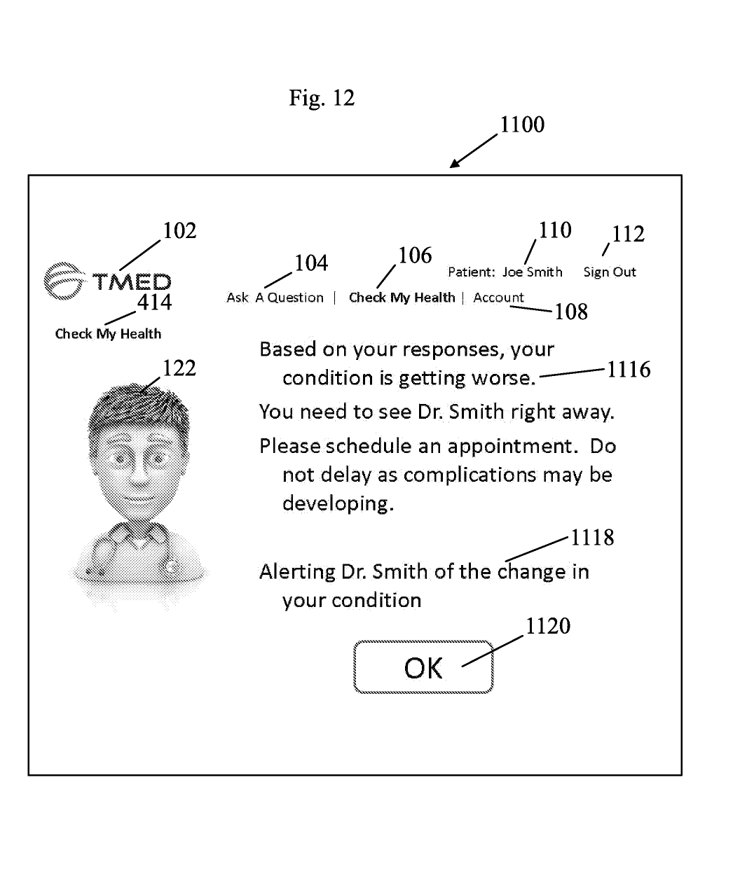

FIG. 12 is an eleventh image 1100 which may be produced on a computer monitor of the apparatus 1 of FIG. 1, in accordance with an embodiment of the present invention. For example, the eleventh image 1100 may be produced on a computer monitor of the interactive health avatar user interface 28 by a computer program executed by a computer processor of the interface 28 and stored in computer memory of the interface 28. The eleventh image 1100 may also be produced by the health avatar artificial intelligence system 26 through a computer program executed by a computer processor of the health avatar artificial intelligence system 26, and stored on computer memory of the system 26. The eleventh image 1100 may be provided to the interface 28 by the system 26, through a communications link which may include the internet.

Referring to FIG. 12, the eleventh image 1100 includes fields 102, 104, 106, 108, 110, 112, 414, 122, 1116, 1118, and 1120. Fields 102, 104, 106, 108, 110, 112, and 122 may be similar to or the same as the same numbered fields in FIG. 1. Field 414 may be similar to or the same as the same numbered field in FIG. 4.

Field 1116 includes text which provides instructions to a patient based on the patient's inputs provided through interface 28 to the system 26. A computer processor of the system 26 may provide the instructions of field 1116 based on the patient's inputs. Field 1118 notifies the patient or user that a Dr. is being alerted. The system 26 may include a computer processor which is programmed to make a telephone call, send an email, or send a text message to a Doctor to alert the doctor regarding a patient's condition in response to the patient's inputs provided through interface 28. Field 1120 includes an enclosed area or box and the text “OK”. A computer processor of the interface 28 may be programmed to process the selection of the field 1120 supply data relating thereto, to the system 26 via a communications link such as including the internet. The computer processor of the interface 28 may execute a web page or program relating to image 1100, which is provided from or downloaded from system 26. The health avatar artificial intelligence system 26, in at least one embodiment, utilizes the patients response, and data from the Health Rules Database 32, to determinate that the alerts to physicians was necessary.

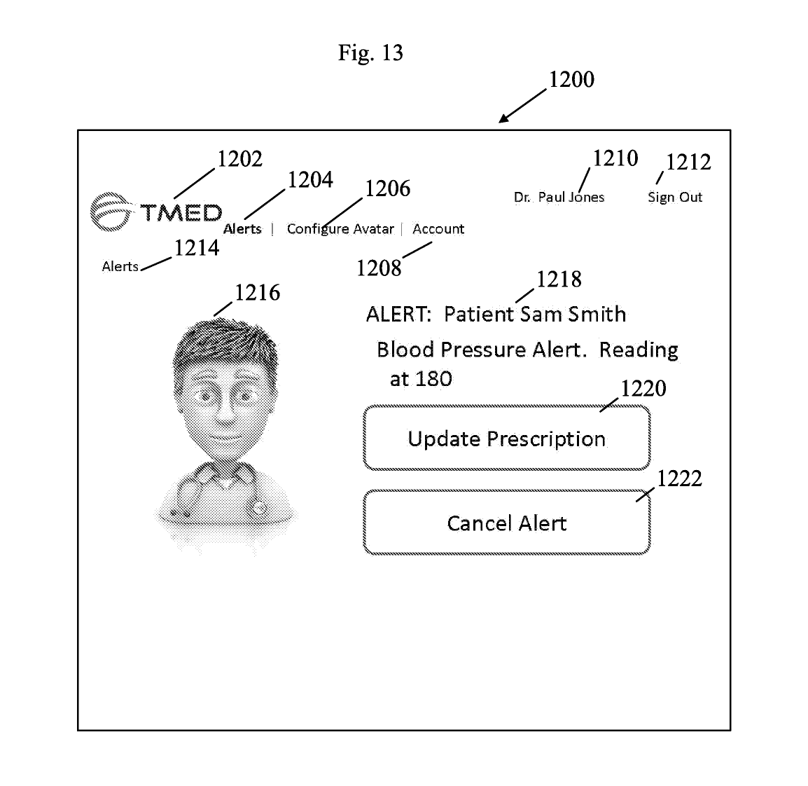

FIG. 13 is an twelfth image 1200 which may be produced on a computer monitor of the apparatus 1 of FIG. 1, in accordance with an embodiment of the present invention. For example, the twelfth image 1200 may be produced on a computer monitor of the interactive health avatar physician interface 30 shown in FIG. 1 by a computer program executed by a computer processor of the physician interface 30 and stored in computer memory of the interface 30. The twelfth image 1200 may also be produced by the health avatar artificial intelligence system 26 through a computer program executed by a computer processor of the health avatar artificial intelligence system 26, and stored on computer memory of the system 26. The twelfth image 1200 may be provided to the physician interface 30 by the system 26, through a communications link which may include the internet.

Referring to FIG. 13, the twelfth image 1200 includes fields 1202, 1204, 1206, 1208,1210, 1212, 1214, 1216, 1218, 1220, and 1222.

Field 1202 includes text “TMED” and a logo for the company running a web site provided through system 26 which provides image or web page 1200. Field 1204 includes text “Alerts” and when selected using a computer interactive device of the physician interface 30, causes the image or web page 1200 to appear on a computer monitor of the physician interface 30. Field 1206 can be selected to display a screen, image or web page on the computer monitor of the interface 30 which allows a physician to configure patient interaction rules for the avatar to follow for every patient. Unique interaction rules may be configured for each patient.

Field 1208 can be selected to display a screen, image or web page for a physician's account regarding the use of the system 26. Field 1210 displays text “Dr. Paul Jones” showing the physician that this refers to. Field 1212 can be selected using a computer interactive device of the physician interface 30 to sign out of the web site provided by system 26. Field 1214 shows that the current screen, image or web page 1200 refers to “Alerts”. Field 1216 displays the avatar or graphic image used for Dr. Paul Jones in this exampled. Field 1218 displays patient information provided by system 26 to the physician's interface 30 for Dr. Jones. Fields 1220 and 1222 shows enclosed areas or boxes with text. The physician can select either field 1220 or field 1222 to update a prescription or cancel an alert respectively, using a computer interactive device of interface 30, which then supplies data or information relating thereto to system 26 via a communications link such as the internet.

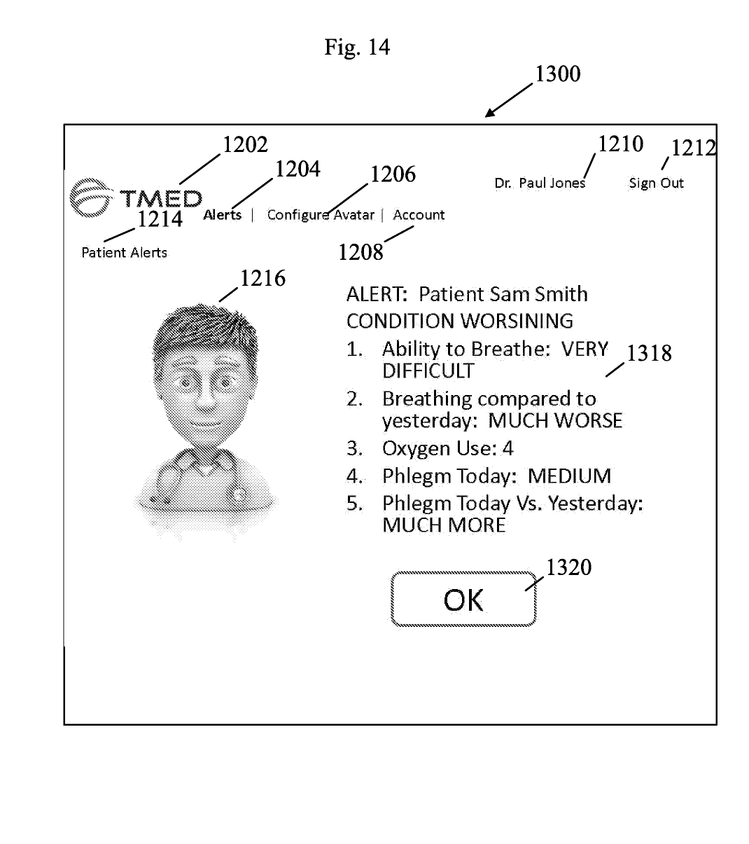

FIG. 14 is an thirteenth image 1300 which may be produced on a computer monitor of the apparatus 1 of FIG. 1, in accordance with an embodiment of the present invention. For example, the thirteenth image 1300 may be produced on a computer monitor of the interactive health avatar physician interface 30 shown in FIG. 1 by a computer program executed by a computer processor of the physician interface 30 and stored in computer memory of the interface 30. The thirteenth image 1300 may also be produced by the health avatar artificial intelligence system 26 through a computer program executed by a computer processor of the health avatar artificial intelligence system 26, and stored on computer memory of the system 26. The thirteenth image 1300 may be provided to the physician interface 30 by the system 26, through a communications link which may include the internet.

Referring to FIG. 14, the thirteenth image 1300 includes fields 1202, 1204, 1206, 1208, 1210, 1212, 1214, 1216, 1318, and 1320. Fields 1202, 1204, 1206, 1208, 1210, 1212, 1214, and 1216 may be similar to or the same as the same numbered fields in FIG. 13.

Field 1318 displays patient information provided by system 26 to the physician's interface 30 for Dr. Jones. Field 1320 an includes enclosed area or box with text “OK”. The physician can select field 1320 to indicate confirmation of having reviewed the patient alert.

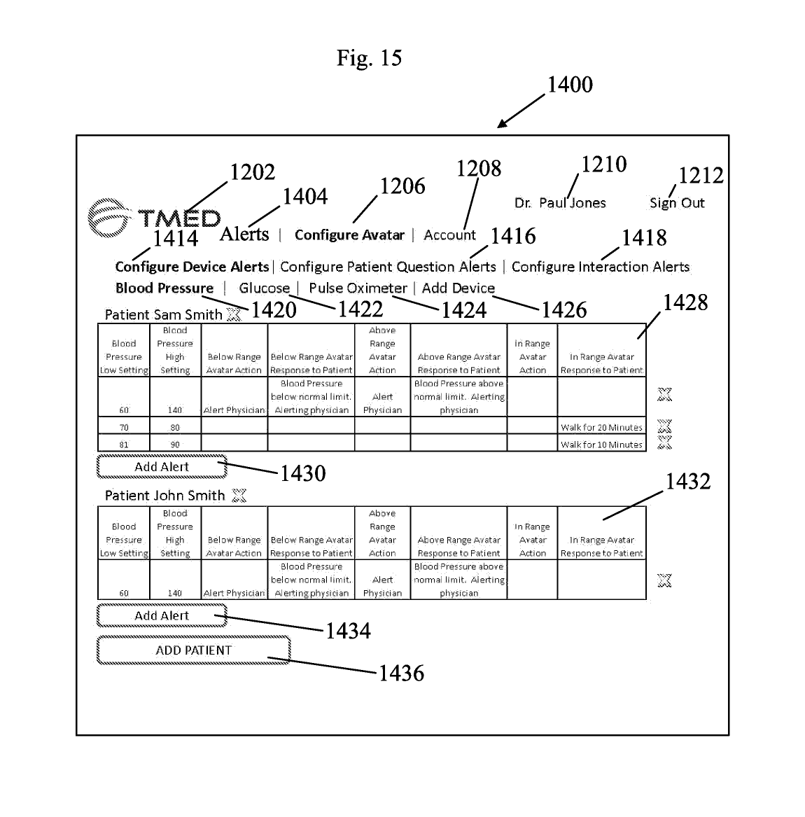

FIG. 15 is an fourteenth image 1400 which may be produced on a computer monitor of the apparatus 1 of FIG. 1, in accordance with an embodiment of the present invention. For example, the fourteenth image 1400 may be produced on a computer monitor of the interactive health avatar physician interface 30 shown in FIG. 1 by a computer program executed by a computer processor of the physician interface 30 and stored in computer memory of the interface 30. The fourteenth image 1400 may also be produced by the health avatar artificial intelligence system 26 through a computer program executed by a computer processor of the health avatar artificial intelligence system 26, and stored on computer memory of the system 26. The fourteenth image 1400 may be provided to the physician interface 30 by the system 26, through a communications link which may include the internet.

Referring to FIG. 15, the fourteenth image 1400 includes fields 1202, 1404, 1206, 1208, 1210, 1212, 1414, 1416, 1418, 1420, 1422, 1424, 1426, 1428, 1430, 1432, 1434, and 1436. Fields 1202, 1206, 1208, 1210, and 1212 may be similar to or the same as the same numbered fields in FIG. 14.

Field 1404 displays text “Alerts” which has been highlighted indicating that it has been selected and the web page for viewing patient alerts is now being presented as depicted in FIG. 14. Field 1414 displays text “Configure Device Alerts” and can be selected to configure device range setting and alerts for patient monitoring devices. Field 1416 includes text “Configure Patient Question Alerts” and can be selected to create questions, range of responses, and alerts action to take for each response, that the avatar will present to the patient. Field 1418 includes text “Configure interaction Alerts” and can be selected to configure alerts which are based on two or more events occurring.

Field 1420 includes text “Blood Pressure” which is in bold, indicates that the blood pressure page is currently loaded where the physician can add patient alerts specific to blood pressure device readings. Field 1422 includes text “Glucose” and can be selected to load a web page where the physician can configure glucose meter device ranges for alerts base on glucose meter device readings. Field 1424 includes text “Pulse Oximeter” and can be selected to do what to load a web page where the physician can configure pulse oximeter device ranges for alerts base on pulse oximeter device readings. Field 1426 includes text “Add Device” and can be selected to load a web page where the physician may add a new type of device and configure setting for alerts base on that device readings. Field 1428 is a table of various medical information for a patient, in this case “Patient Sam Smith”. The large “X” symbols show next to rows of table or field 1428 and next to rows of table or field 1432 in FIG. 15 can be selected to delete a row of information in the particular row. Field 1430 includes an enclosed area or box with text “Add Alert” which can be selected, such as by using a computer interactive device of interface 30, to add another data row to the table or field 1428 where the physician can add another blood pressure monitoring device alert and corresponding entries across the columns for this patient.

Field 1432 is a table of various medical information for “Patient John Smith”. Field 1434 includes an enclosed area or box with text “Add Alert” which can be selected by a doctor using a computer interactive device of interface 30 which can be selected to add another data row to the table or field 1432 where the physician can add another blood pressure monitoring device alert and corresponding entries across the columns for this patient. Field 1436 includes an enclosed area or box with text “Add Patient” which can be selected by a doctor using a computer interactive device, such as a computer mouse, computer touch screen or computer keyboard of interface 30 to add a patient to configure blood pressure alerts for.

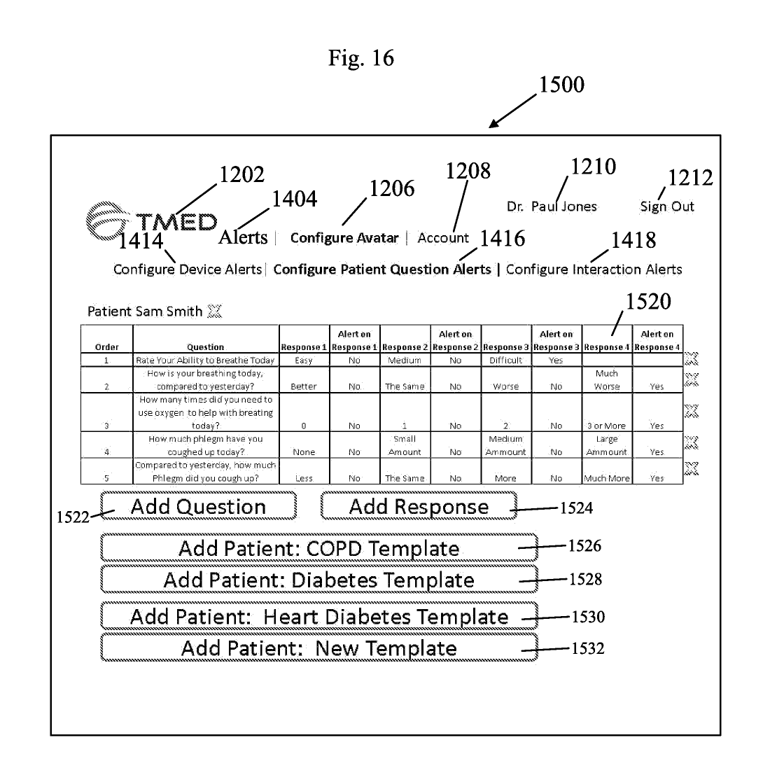

FIG. 16 is an fifteenth image 1500 which may be produced on a computer monitor of the apparatus 1 of FIG. 1, in accordance with an embodiment of the present invention. For example, the fifteenth image 1500 may be produced on a computer monitor of the interactive health avatar physician interface 30 shown in FIG. 1 by a computer program executed by a computer processor of the physician interface 30 and stored in computer memory of the interface 30. The fifteenth image 1500 may also be produced by the health avatar artificial intelligence system 26 through a computer program executed by a computer processor of the health avatar artificial intelligence system 26, and stored on computer memory of the system 26. The fifteenth image 1500 may be provided to the physician interface 30 by the system 26, through a communications link which may include the internet.

Referring to FIG. 16, the fifteenth image 1500 includes fields 1202, 1404, 1206, 1208, 1210, 1212, 1414, 1416, 1418, 1520, 1522, 1524, 1526, 1528, 1530, and 1532. Fields 1202, 1404, 1206, 1208, 1210, 1212, 1414, 1416, and 1418 may be similar to or the same as the same numbered fields in FIG. 15.

Field 1520 is a table of various medical questions, preconfigured responses to select from, and alert actions to take for each preconfigured response, for a patient, in this case “Patient Sam Smith”. The “X” symbols in FIG. 16, mean delete this patient entry from the system database. Fields 1522-1532 include enclosed areas or boxes with text which can be clicked on to load a web page for the physician or healthcare provider to enter the initial set of questions and possible patient response that the health avatar will use to interact with the patients. Fields 1526-1532 enable the physician or healthcare provider to create a general template of questions and possible responses that can be used to simplify the process of adding a new patient with a specific condition to the system. Field 1522 can be selected, by using a computer interactive device of interface 30, to add a new row to the field or table 1520 for a patient question where the physician enters an additional question, possible responses for that question, and alert actions to take for each possible response. Field 1524 can be selected, by using a computer interactive device of interface 30 to add two new columns to the field or table 1520, where the physician can add another possible patient response and alert action to take if patient selects this response. This action adds two new column for every question for the patient. Not all questions will use every column available for possible responses and alert actions.

Field 1526 can be selected by using a computer interactive device of interface 30 to add a predetermined set of questions, range of possible responses, and alert action for each response for a COPD (chronic obstructive pulmonary disease) patient, to the table or field 1520 in FIG. 16. The physician will select the patient's name on the form, and the questions, possible responses, and alerts will be automatically loaded for that patient. This reduces the administrative time required to input questions for a COPD patient.

Field 1528 can be selected by using a computer interactive device of interface 30 to add a predetermined set of questions, possible responses, and alert action for each response for a diabetes patient, to the table or field 1520 in FIG. 16. In at least one embodiment, the physician will select the patient's name on the form, and the questions and possible responses will be automatically loaded for that patient. This reduces the administrative time required to input questions for a diabetes patient.

Field 1530 can be selected by using a computer interactive device on interface 30 to add a predetermined set of questions, possible responses, and alert action for each response for a heart disease patient. The physician will select the patient's name on the form, and the questions and possible responses will be automatically loaded for that patient. This reduces the administrative time required to input questions for a heart disease patient.

Field 1532 can be selected by using a computer interactive device on interface 30 to load a web page where a physician can create a health condition template of questions, possible responses, and alert actions for each possible action.

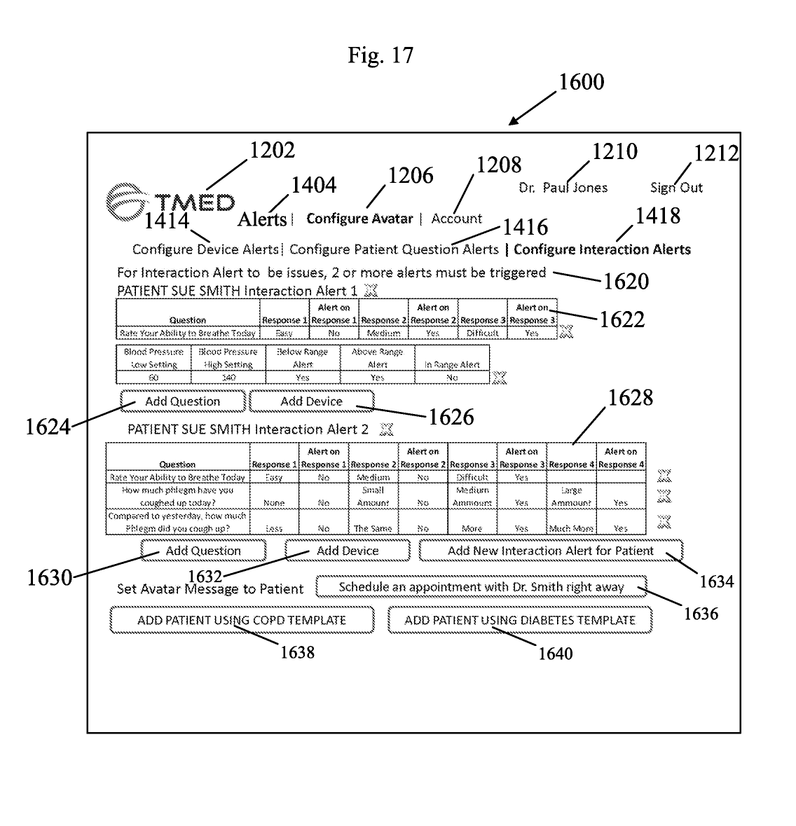

FIG. 17 is an sixteenth image 1600 which may be produced on a computer monitor of the apparatus 1 of FIG. 1, in accordance with an embodiment of the present invention. For example, the sixteenth image 1600 may be produced on a computer monitor of the interactive health avatar physician interface 30 shown in FIG. 1 by a computer program executed by a computer processor of the physician interface 30 and stored in computer memory of the interface 30. The sixteenth image 1600 may also be produced by the health avatar artificial intelligence system 26 through a computer program executed by a computer processor of the health avatar artificial intelligence system 26, and stored on computer memory of the system 26. The sixteenth image 1600 may be provided to the physician interface 30 by the system 26, through a communications link which may include the internet.

Referring to FIG. 17, the sixteenth image 1600 includes fields 1202, 1404, 1206, 1208, 1210, 1212, 1414, 1416, 1418, 1620, 1622, 1624, 1626, 1628, 1630, and 1632. Fields 1202, 1404, 1206, 1208, 1210, 1212, 1414, 1416, and 1418 may be similar to or the same as the same numbered fields in FIG. 17.

Field 1418 is in bold on this page, indicating that the web page to Configure Interaction Alerts is now loaded. Field 1620 is a message indicating providing instruction to the physician. Fields 1622 and 1628 are tables of various alert settings for a patient, in this case “Patient Sue Smith”. In at least one embodiment, for the interaction type of alert to be triggered, two or more alerts have to be triggered for a patient. The “X”'s next to rows of tables or fields 1622, and 1628 can be selected to delete a row of information of the particular table or field.

Field 1622 is a table of interaction alerts, in this case for Sue Smith. In at least one embodiment, If two or more alerts identified in this table are triggered, meaning that this activity has happened, then an alert message will be sent to Sue Smith's physician or health care provider, such as to interactive health avatar physician interface 30 by health avatar artificial intelligence system 26.

Field 1624 can be selected by a computer interactive device of interface 30 to add a question based alert to table 1622, where a physician enters a question, possible responses, and alert status for each response, in this case for patient “Sue Smith”.

Field 1626 can be selected, by a user using a computer interactive device of interface 30, to add a device based alert to table 1622, where the physician adds a device range and alert status if the patient is below range, in range, or above range. An alert can be established for each possible range status, in this case for patient “Sue Smith”.

Table 1628 is another table of interaction alerts, in this case also for Sue Smith. If two or more alerts identified in this table are triggered, meaning that this activity has happened, then an alert message will be sent to Sue Smith's physician or health care provider from system 26, such as to interface 30 as depicted in FIG. 14. There can be multiple interaction alerts defined for every patient. Field 1630 can be selected to add a question based alert for the second interaction alert table 1628, where physician enters a question, possible responses, and alert status for each response. Field 1632 can be selected to add a device based the second interaction alert table 1628, where the physician adds a device range and alert status if the patient is below range, in range, or above range. An alert can be established for each possible range status.

Field 1634 create a new table, such as 1622 and 1628, that can be populated with question and device alerts. Field 1638 creates an alert interaction table for a patient, such as 1622 and 1628, that is pre-populated with questions and device alerts that are used for treating patients with COPD.

Field 1640 creates an alert interaction table for a patient, such as 1622 and 1628, that is pre-populated with questions and device alerts that are used for treating patients with diabetes. Other templates may be created or utilized for treating other medical conditions.

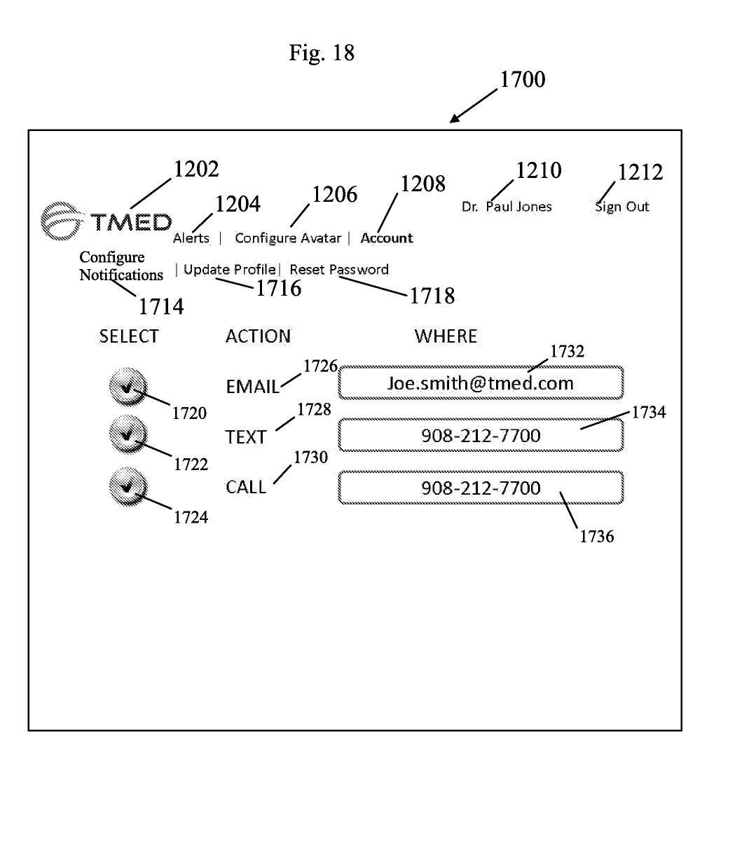

FIG. 18 is an seventeenth image 1700 which may be produced on a computer monitor of the apparatus 1 of FIG. 1, in accordance with an embodiment of the present invention. For example, the seventeenth image 1700 may be produced on a computer monitor of the interactive health avatar physician interface 30 shown in FIG. 1 by a computer program executed by a computer processor of the physician interface 30 and stored in computer memory of the interface 30. The seventeenth image 1700 may also be produced by the health avatar artificial intelligence system 26 through a computer program executed by a computer processor of the health avatar artificial intelligence system 26, and stored on computer memory of the system 26. The seventeenth image 1700 may be provided to the physician interface 30 by the system 26, through a communications link which may include the internet.

Referring to FIG. 18, the seventeenth image 1700 includes fields 1202, 1204, 1206, 1208, 1210, 1212, 1714, 1716, 1718, 1720, 1722, 1724, 1726, 1728, 1730, 1732, 1734, and 1736. Fields 1202, 1204, 1206, 1208, 1210, and 1212 may be similar to or the same as the same numbered fields in FIG. 13.

Field 1714 is a sub-page under “Account” field 1208. “Account” field 1208 is highlighted indicating that the account menu item has been selected. “Configure Notifications” field 1714, is highlighted indicating that the sub-page Configure Alerts is loaded under the “Account” page or field. This page allows a physician to configure how to be alerted when necessary.

Field 1716 when selected using a computer interactive device of interface 30 loads a web page for display on a computer monitor of interface 30 where a physician can update basic information such as contact information.

Field 1718, when selected, using a computer interactive device of a interface 30 loads a web page on the interface 30 where a physician can reset passwords.

Field 1720 is a check box/button that a physician can select using interface 30 to indicate send alerts via email, 1726 to the email address entered in 1723.

Field 1722 is a check box/button that a physician can select using interface 30 to indicate send alerts via text, 1728 to the phone number entered in 1734.

Field 1724 is a check box/button that a physician can select using interface 30 to indicate send alerts via telephone call, to the phone number entered in 1736.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

I claim:

1. A method comprising
receiving an input via a user interactive device, wherein the input provides data concerning a health question about a user;
supplying the data concerning the health question about the user to a computer processor;
supplying information concerning the user's health from a plurality of medical devices to the computer processor;
using the computer processor to determine an answer to the health question about the user by using the data concerning the health question about the user and the information concerning the user's health supplied from the plurality of medical devices; and
displaying the answer to the health question on a computer display of the user interactive device.

2. The method of claim 1 wherein
the user interactive device is a smart phone.

3. The method of claim 1 wherein
the answer to the health question about the user is displayed along with an avatar, so that the avatar appears to be providing the answer to the health question.

4. The method of claim 1 wherein
the answer to the health question is displayed along with a first field that is configured to be selected by the user to provide feedback to the computer processor via the user interactive device to indicate that the health question has been answered to the satisfaction of the user and a second field that is configured to be selected by the user to provide feedback to the computer processor via the user interactive device to indicate that the health question has not been answered to the satisfaction of the user.

5. The method of claim 4 wherein
using the feedback from the user interactive device indicating that the health question has not been answered to the satisfaction of the user to modify rules stored in a computer memory concerning how answers to questions about the user's health are determined by the computer processor.

6. The method of claim 1 wherein
the data concerning the health question of the user includes data concerning a request to generally check the health of the user;
and wherein the answer to the health question includes information concerning a plurality of health parameters.

7. The method of claim 6 wherein
the plurality of health parameters includes results obtained from a plurality of health monitoring devices.

8. The method of claim 6 wherein
the plurality of health parameters includes blood pressure.

9. The method of claim 6 wherein
the plurality of health parameters includes a drug prescription for the user.

10. The method of claim 6 wherein
the information concerning the plurality of health parameters includes a message indicating that the user should provide feedback as to the level of a particular health condition; and further comprising
displaying a first plurality of fields for indicating levels of the particular health condition, wherein each of the first plurality of fields is configured to be selected by the user to provide information to the computer processor via the user interactive device on a level of the particular health condition.

11. The method of claim 10 further comprising
providing a recommendation message from the computer processor to the user interactive device based on which of the first plurality of fields for indicating levels of the particular health condition is selected by the user; and
wherein the recommendation message provides information concerning the particular health condition to the user.

12. The method of claim 11 further comprising
using the computer processor to notify, via a communications link, a health care professional, a name of the user and the particular health condition, and the fact that a recommendation message has been provided to the user interactive device.

13. The method of claim 12 further comprising
displaying on a health care professional interactive device data concerning the particular health, and the name of the user, and the fact that the recommendation message has been provided to the user interactive device.

14. The method of claim 10 further comprising
displaying on a health care professional interactive device a second plurality of fields each of which can be selected to place the user in a category for treatment for the particular health condition; and
wherein each of the second plurality of fields is configured to be selected by a health care professional to display predetermined data relating to the particular health condition.

15. The method of claim 14 wherein
the predetermined data includes questions which are sent to the user interactive device by the computer processor in response to selection of a field of the second plurality of fields.

16. An apparatus comprising
a computer processor;
a computer memory;
and a computer display;
wherein the computer processor is programmed to:
receive data concerning a health question about a user;
receive information concerning the health of the user from a plurality of medical devices;
to determine an answer to the health question about the user by using the data concerning the health question about the user and the information concerning the health of the use supplied from the plurality of medical devices; and
to display the answer to the health question on the computer display.
